(19) United States
(12) Patent Application Publication
(10) Pub. No.: US 2024/0050113 A1
(43) Pub. Date: Feb. 15, 2024

(54) STEERABLE AND EXPANDABLE ASPIRATION CATHETER

(71) Applicant: DAEGU GYEONGBUK INSTITUTE OF SCIENCE AND TECHNOLOGY, Daegu (KR)

(72) Inventors: Hong Soo CHOI, Daegu (KR); Hak Joon LEE, Daegu (KR); Jin Young KIM, Daegu (KR)

(73) Assignee: DAEGU GYEONGBUK INSTITUTE OF SCIENCE AND TECHNOLOGY, Daegu (KR)

(21) Appl. No.: 18/446,036

(22) Filed: Aug. 8, 2023

(30) Foreign Application Priority Data
Aug. 9, 2022 (KR) 10-2022-0098983

(51) Int. Cl. A61B 17/221; A61B 34/00

(52) U.S. Cl. CPC A61B 17/221; A61B 34/73; A61B 2017/00327

(57) ABSTRACT

Provided is an aspiration catheter having a tubular shape and used to remove an embolus or a thrombus by being inserted into a blood vessel, and more particularly, a steerable and expandable aspiration catheter which may apply an effective aspiration force to an embolus or a thrombus by steering a distal tip of the catheter in a three-dimensional direction and expanding a diameter of the tip.

### CROSS-REFERENCE TO RELATED APPLICATIONS

This application claims priority under 35 U.S.C. § 119 to Korean Patent Application No. 10-2022-0098983, filed on Aug. 9, 2022, in the Korean Intellectual Property Office, the disclosure of which is incorporated herein by reference in its entirety.

### TECHNICAL FIELD

The following disclosure relates to an aspiration catheter having a tubular shape and used to remove an embolus or a thrombus by being inserted into a blood vessel, and more particularly, to a steerable and expandable aspiration catheter which may apply an effective aspiration force to an embolus or a thrombus by steering a distal tip of the catheter in a three-dimensional direction and expanding a diameter of the tip.

### BACKGROUND

A catheter is a medical instrument inserted into a target tissue such as various organs (e.g., bile duct or heart) through a body cavity, a luminal cavity, a blood vessel, or the like.

Among these catheters, an aspiration and thrombus collection catheter or device may be generally used in performing a mechanical thrombectomy for intervention in the blood vessel when a patient suffers from a disease such as an acute ischemic stroke (AIS), a myocardial infarction (MI), or a pulmonary embolism (PE).

In a state where a tip of the catheter, which is its end, is brought to be close to the thrombus in order to remove the thrombus, the thrombus may then be pulled to be removed by withdrawing the catheter while the thrombus is maintained to be in close contact with the tip through a strong aspiration force.

Here, the larger cross-sectional area where the thrombus and the tip of the catheter are in contact with each other, that is, the larger diameter of the tip of the catheter, the more advantageous it is to effectively remove the thrombus. In addition, the aspiration force of the catheter may be maintained at its maximum to easily remove the thrombus only when the thrombus and the tip of the catheter are in contact with each other in a state where a vascular axis of the thrombus and a central axis of the tip of the catheter are positioned in a straight line. The reason is that the aspiration force may be reduced by being offset by a reaction force with an inner wall of the blood vessel when the thrombus and the tip of the catheter are in contact with each other in a state where the vascular axis of the thrombus and the central axis of the tip of the catheter are tilted rather than being positioned in a straight line.

The following conventional techniques have been respectively disclosed: a technique of improving an aspiration efficiency of the catheter by aspirating the thrombus after expanding the diameter of the tip of the catheter by expanding the tip while the tip is close to the thrombus; or a technique of improving an aspiration efficiency of the catheter by aspirating the thrombus in a state where a direction of the tip of the catheter is steered to position the vascular axis of the thrombus and the central axis of the tip of the catheter to be in a straight line.

However, a technique of implementing only one of the functions of expanding or steering the tip of the catheter is conventionally known. It is impossible to simultaneously implement the functions of steering and expanding the tip through a simple combination of the known techniques, and the catheter may have a complicated configuration and an increased size even if implemented. Therefore, there is a need for the development of a technique of simultaneously applying the expansion or steering of the tip of the catheter.

### SUMMARY

An embodiment of the present disclosure is directed to providing a steerable and expandable aspiration catheter using one mechanism in which a distal tip of the catheter is steered by a pulling displacement of a plurality of wires, which are its drive parts, and simultaneously, a diameter of the tip is expanded when the plurality of wires are simultaneously pulled to the same displacement.

Another embodiment of the present disclosure is directed to providing an aspiration catheter which may control a direction of its tip by a magnetic torque of a magnetic body positioned at an end of the tip, and simultaneously expand the tip by adjusting a strength of a magnetic field of the magnetic body.

In one general aspect, a steerable and expandable aspiration catheter includes: a body; a steering part positioned at an end of the body and steering the end; an expanding part connected to an end of the steering part and expanding a diameter of the end; and a power unit providing power for the steering and the expansion, wherein the steering part includes a first body having a circular ring shape and a leg connecting the first body and the expanding part to each other, the expanding part includes a second body having a circular ring shape and a plurality of tip parts each having an annular shape, each protruding outward from the second body, and arranged in a radial direction, and the power unit includes a plurality of wires, each wire being connected to each tip part, and controls the steering of the tip part or expands a diameter of the tip part based on a displacement of the wire pulling the tip part in a direction opposite to an end of the catheter.

The steering part may include a pair of a 1-1-th body and a 1-2-th body each having a circular ring shape and spaced apart from each other, a first leg connecting the 1-1-th body and the 1-2-th body to each other, and a second leg connecting the 1-2-th body and the second body to each other, one pair of the first legs or one pair of the second legs may be disposed to face each other, and a first line connecting the pair of first legs to each other and a second line connecting the pair of second legs to each other may be orthogonal to each other.

The power unit may control the steering of the tip part of the catheter by generating the displacement between one pair of wires disposed on one side of the wires and one pair of wires disposed on the other side of the wires.

The power unit may expand the diameter of the tip part as each tip part is bent outward in the radial direction when all of the wires are pulled to generate no displacement difference between the respective wires.

In the catheter, the diameter of the tip part may be controlled by a displacement as much as the amount that the wire is pulled when all of the wires are pulled.

The tip part may include a tip part body having an annular shape and protruding outward from the second body while being spaced apart therefrom by a certain distance, and a tip part supporter made of a flexible material, having a linear shape, connecting the tip part body and the second body to each other, and supporting the tip part body.

In another general aspect, a steerable and expandable aspiration catheter includes: a body; a steering part made of a magnet and positioned at an end of the body; an expanding part made of a combination of a plurality of magnets and positioned at an end of the steering part; and a magnetic field generating unit generating a magnetic field around the steering part and the expanding part, wherein the steering part controls steering of the steering part based on a direction of the magnetic field, and the expanding part controls a diameter of an end of the catheter based on a strength of the magnetic field.

The steering part may be a circular base magnet and has polarities disposed in a length direction of the catheter, and the base magnet may steer the end of the catheter based on the direction of the magnetic field.

The expanding part may include a tip magnet part disposed outside an end of the base magnet to be spaced apart therefrom and a leg connecting the tip magnet part and the steering part to each other, the tip magnet part may be a combination of a plurality of arc-shaped magnets, have polarities disposed in a circumferential direction, and may be displaced outward in a radial direction based on a strength of the magnetic field generated around the expanding part to expand a diameter of the end of the catheter.

The catheter may further include a catheter for pressure measurement positioned on the outside of the body and capable of measuring a surrounding pressure.

### BRIEF DESCRIPTION OF THE DRAWINGS

FIG. 1 is a perspective view of a tip part of a catheter according to a first embodiment of the present disclosure.

FIG. 2 is a perspective view of an operation state of the catheter when steering its tip part according to a first embodiment of the present disclosure.

FIG. 3 is a perspective view of an operation state of the catheter when expanding itstip part according to a first embodiment of the present disclosure.

FIG. 4 is a side view of the tip part of the catheter according to a first embodiment of the present disclosure.

FIG. 5 is a perspective view of a tip part of a catheter according to a second embodiment of the present disclosure.

FIG. 6 is a perspective view of an operation state of the catheter when steering its tip part according to a second embodiment of the present disclosure.

FIG. 7 is a schematic view of an operation state of the catheter when expanding its tip part according to a second embodiment of the present disclosure.

FIG. 8 is a schematic view of a catheter according to a third embodiment of the present disclosure.

### DETAILED DESCRIPTION OF EMBODIMENTS

Hereinafter, embodiments of the present disclosure are described in detail with reference to the accompanying drawings.

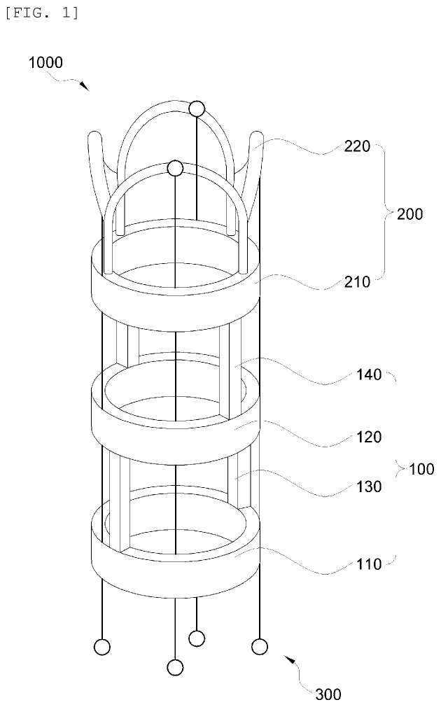

FIG. 1 is a perspective view of a tip part of a catheter 1000 according to a first embodiment of the present disclosure. As shown in the drawing, the catheter 1000 may include a steering part 100 steering its tip part, an expanding part 200 expanding or reducing a diameter of the tip part, and a power unit 300 providing power for the steering and the expansion. Although not shown in the drawing, a catheter cover made of a thin film of a flexible material may be positioned on an outer surface of the catheter 1000.

In more detail, the steering part 100 may include a pair of a 1-1-th body 110 and a 1-2-th body 120 each having a circular ring shape and spaced apart from each other. The 1-1-th body 110 and the 1-2-th body 120 may be connected to each other through a first leg 130, and the 1-2-th body 120 and a second body 210 of the expanding part 200 may be connected to each other through a second leg 140. The first leg 130 and the second leg 140 may each be made of a flexible material and may steer an end of the catheter 1000 by being driven by the power unit 300. Each pair of the first legs 130 and that of the second legs 140 may be disposed to face each other. In addition, the first leg 130 and the second leg 140 may not be disposed at the same position in an axial direction of the catheter 1000, and may be spaced apart from each other at 90 degree intervals. In other words, a first imaginary line connecting upper or lower ends of the pair of first legs 130 with each other and a second imaginary line connecting the upper or lower ends of the pair of second legs 140 with each other may be orthogonal to each other rather than coinciding with each other when viewed from the axial direction of the catheter 1000.

Therefore, either one of the pair of first legs or either one of the pair of second legs may be bent when the catheter 1000 is steered in an x direction, and the other may be bent when the catheter 1000 is steered in a y direction, such that the end of the catheter 1000 is steered in the x-axis direction or the y-axis direction.

The expanding part 200 may include the second body 210 connected to the 1-2-th body 120 through the second leg 140 and an annular tip part 220 protruding outward from the second body 210. The plurality of tip parts 220 may be arranged along a circumference of the second body 210 in a radial direction. An end of the tip part 220 may be connected to the power unit 300 to be steered based on an operation of the power unit 300, or the end of the tip part 220 may be bent outward in the radial direction to expand a diameter of the end of the catheter 1000.

The power unit 300 may include a plurality of wires, each passing through the 1-1-th body 110, 1-2-th body 120, and the second body 210, and having each end fixed to the end of the tip part 220. The power unit 300 as described above may steer or expand the tip part 220 based on a displacement of each wire pulling the tip part 220 in a direction opposite to the end of the catheter 1000. That is, when some of the plurality of wires are pulled to have a displacement difference with other wires, the tip part 220 on a side where the wires are being pulled may be steered toward the side where the wire is being pulled, and when all of the plurality of wires are pulled to the same displacement, the tip part 220 may be bent outward in the radial direction to expand the diameter of the tip part 220.

Hereinafter, the end steering and diameter expansion of the catheter 1000 having the above configuration are described in more detail with reference to the drawings.

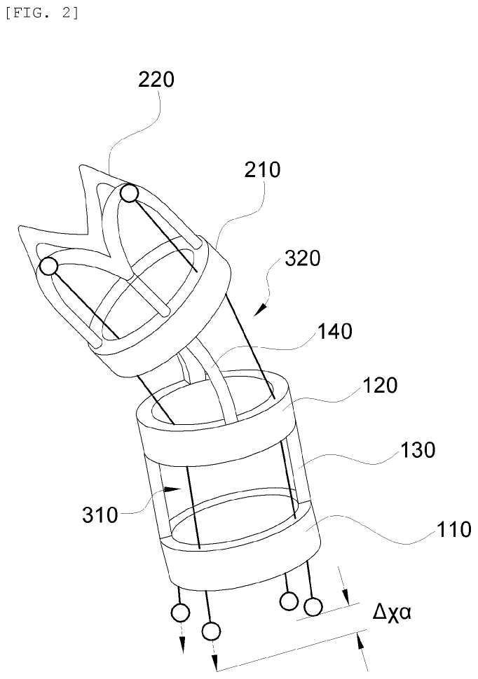

FIG. 2 is a perspective view of an operation state of the catheter 1000 when steering the tip part 220 of the catheter according to a first embodiment of the present disclosure.

As shown in the drawing, only a pair of first wires 310 disposed on one side of the power unit 300 may be pulled and a pair of second wires 320 disposed on the other side may be fixed. In this case, the displacement difference may be generated between the first wire 310 and the second wire 320, and the tip part 220 of the catheter 1000 may be steered toward a side where the first wire 310 is positioned by a displacement as much as the amount that the first wire 310 is pulled. That is, a displacement ΔXa may be generated between one pair of first wires 310 and another pair of second wires 320 of the power unit 300. In this case, the steering of the tip part 220 of the catheter 1000 may be controlled by the displacement difference. In more detail, as shown in the drawing, the tip part 220 of the catheter 1000 may be steered toward the side where the pulled pair of first wires 310 are positioned. Further, one pair of first wires 310 disposed on one side may be pulled and one pair of second wires 320 disposed on the other side may be released. In this case, the displacement of the first wire 310 and the displacement of the second wire 320 may be combined with each other to increase the displacement ΔXa and increase a steering speed, thus rapidly steering the tip part 220 of the catheter.

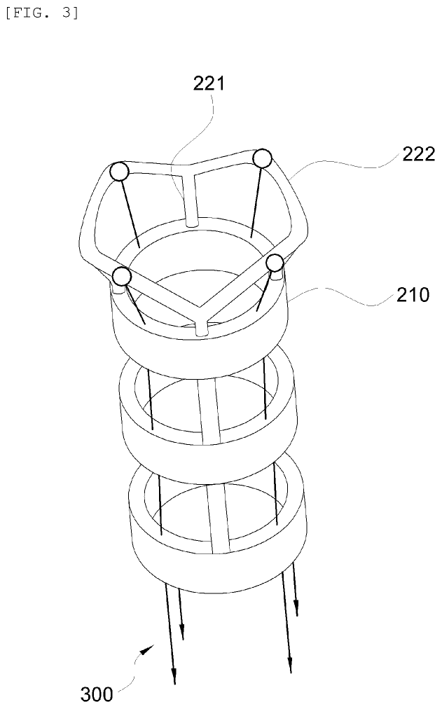

FIG. 3 is a perspective view of an operation state of the catheter 1000 when expanding the tip part 220 of the catheter according to a first embodiment of the present disclosure.

As shown in the drawing, the tip part 220 may include a tip part body 222 having an annular shape and protruding outward from the second body 210 while being spaced apart therefrom by a certain distance, and a tip part supporter 221 having a linear shape, connecting the tip part body 222 and the second body 210 to each other, and supporting the tip part body 222.

All the wires of the power unit 300 of the catheter 1000 may be pulled and no displacement difference between the respective wires may be generated. In this case, the diameter of the tip part 220 may be expanded as the tip part supporter 221 is bent so that each tip part body 222 of the catheter 1000 is expanded outward in the radial direction while the steering of the tip part 220 is maintained.

In addition, a size of the diameter of the tip part 220 may be controlled based on a displacement magnitude of the wire. That is, the diameter of the tip part 220 may be significantly expanded when the displacement of the wire is significant, and the diameter of the tip part 220 may be insignificantly expanded when the displacement of the wire is insignificant. In addition, when all the wires of the power unit 300 are released, the diameter of the tip part 220 may be restored to its original shape as each tip part 220 of the catheter 1000 is restored to be inward in the radial direction.

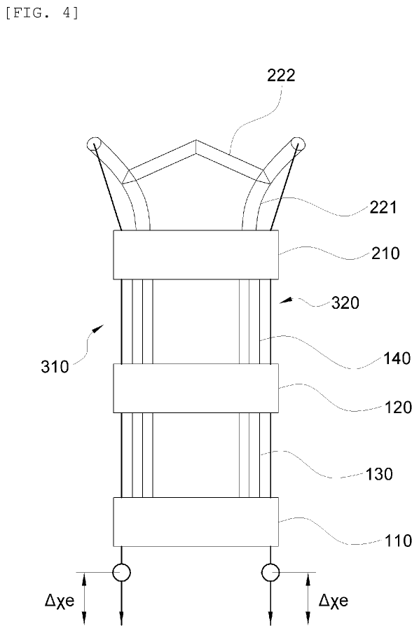

FIG. 4 is a side view of the tip part 220 of the catheter 1000 according to a first embodiment of the present disclosure.

As shown in the drawing, a displacement ΔXe of one pair of wires 310 disposed on one side of the power unit 300 and the displacement ΔXe of the other pair of wires 320 disposed on the other side are the same as each other, that is, no displacement difference may be generated therebetween. In this case, the tip part 220 may be bent outward in the radial direction to thus expand the diameter of the tip part 220, and control the diameter of the tip part 220 by a magnitude of the displacement ΔXe. On the other hand, a difference may be generated between the displacement ΔXe of one pair of wires 310 disposed on one side of the power unit 300 and the displacement ΔXe of the other pair of wires 320 disposed on the other side. In this case, the end of the catheter 1000 may be bent toward a side where the displacement is generated to thus control the steering of the tip part 220.

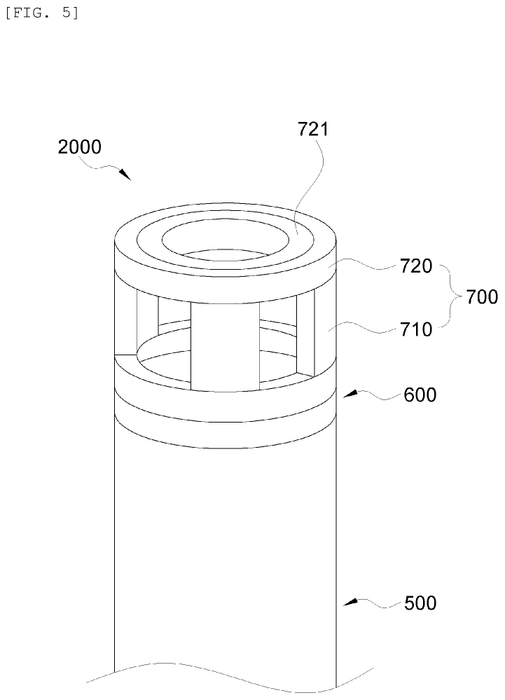

FIG. 5 is a perspective view of a catheter 2000 according to a second embodiment of the present disclosure. As shown in the drawing, the catheter 2000 may include a body 500, a steering part 600 positioned at an end of the body 500, an expanding part 700 positioned at an end of the steering part 600, and although not shown in the drawing, a magnetic field generating unit generating a magnetic field around the steering part 600 and the expanding part 700. And a catheter cover made of a thin film of a flexible material may be positioned on an outer surface of the catheter 2000.

The steering part 600 may be a circular base magnet and have polarities disposed in a length direction of the catheter. That is, the steering part 600 may have either one of the N pole or the S pole that is disposed on one side in the length direction, and the other pole that is disposed on the other side in the length direction.

Therefore, the circular base magnet is aligned according to the direction of the magnetic field generated around the steering unit 600 through the magnetic field generating unit, and the steering of the steering part 600 is controlled to steer an end of the catheter 2000.

Here, the magnetic field generating unit may be configured to apply a magnetic field to the steering part 600 applied through a current by using an external coil system.

In addition, the catheter 2000 may be steered in three dimensions through the steering part 600 when the direction of the magnetic field is controlled in the three dimensions by the external coil system.

In addition, when the magnetic field direction is controlled in three dimensions through the external coil system, it can be configured to enable three-dimensional steering of the end of the catheter 2000 through the steering part 600.

The expanding part 700 may include a tip magnet part 720 disposed outside the end of the steering part 600 to be spaced apart therefrom, and a leg 710 made of a flexible material and connecting the tip magnet part 720 and the steering part 600. The tip magnet part 720 may be a combination of a plurality of arc-shaped magnets 721, and have polarities disposed in a radial direction. That is, the magnet 721 may have either one of the N pole or the S pole that is disposed inward in the radial direction, and the other pole that is disposed outward in the radial direction.

In addition, each magnet 721 may be connected to the steering part 600 through the corresponding leg 710, and displaced outward in the radial direction based on a strength of the magnetic field generated around the expanding part 700 to expand a diameter of the expanding part 700.

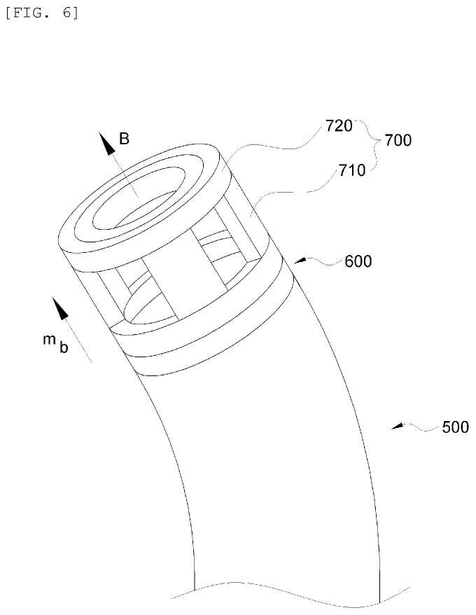

FIG. 6 is a perspective view of an operation state of the catheter 2000 when steering its tip part according to a second embodiment of the present disclosure.

As shown in the drawing, the steering part 600 may control the steering of the tip part of the catheter 2000 in a three-dimensional direction to coincide with a direction of the magnetic field generated around the steering part 600.

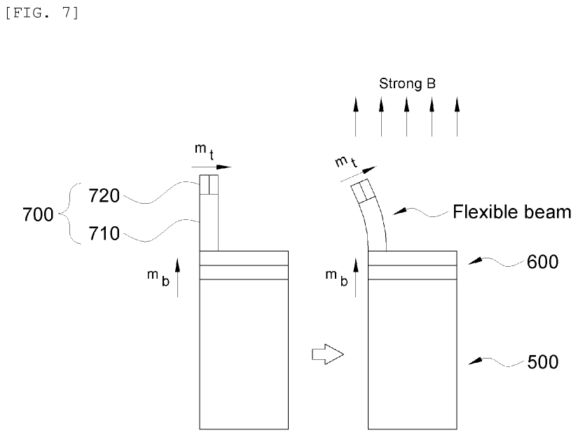

FIG. 7 is a schematic view of an operation state of the catheter 2000 when expanding its tip part according to a second embodiment of the present disclosure.

As shown in the drawing, each magnet of the tip magnet part 721 of the expanding part 700 may be displaced outward in the radial direction based on the strength of the magnetic field generated around the expanding part 700, and an end of the leg 710 that is connected to each magnet may also be bent outward in the radial direction to expand the diameter of the expanding part 700.

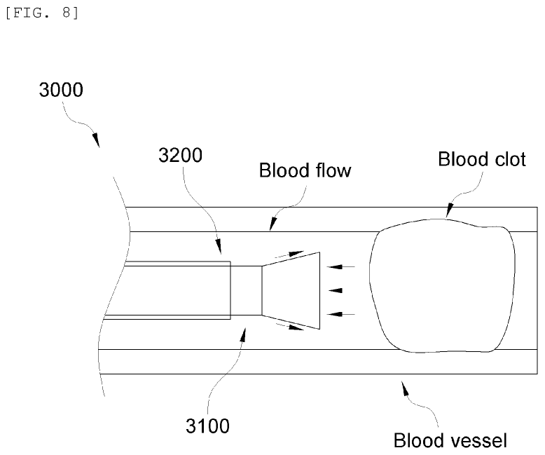

FIG. 8 is a schematic view of a catheter 3000 according to a third embodiment of the present disclosure. As shown in the drawing, the catheter 3000 according to a third embodiment of the present disclosure may include a catheter 3200 for pressure measurement that is positioned on the outside of the body 3100, and connected to an external transducer to measure a pressure. That is, the catheter 3000 may have a double catheter structure in which the body 3100 for thrombus aspiration is positioned inside and the catheter 3200 for pressure measurement is positioned outside.

Therefore, the catheter 3000 of the present disclosure may use the catheter body 3100 for thrombus aspiration and the catheter 3200 for pressure measurement together. Accordingly, the catheter 3000 may measure pressure information of a surrounding blood vessel in real time during catheter aspiration, use the pressure information to determine an expansion degree of a tip of the catheter, and may be used to determine a position of a clogged blood vessel and aspiration efficiency.

As set forth above, the steerable and expandable aspiration catheter of the present disclosure having the above configuration may have the increased aspiration efficiency and prevent loss of the thrombus fragment by locally restricting the blood flow through the tip having expanded diameter and increasing the cross-sectional area where the catheter and the thrombus are in contact with each other during the thrombus aspiration. In addition, the greater aspiration force may be applied to the thrombus even with the same vacuum pressure due to the increased contact cross-sectional area during the thrombus aspiration.

In addition, the catheter may be stably moved even in the narrow and curved blood vessel by steering its tip in the three-dimensional direction, and the greater aspiration force may be applied to the thrombus by aligning the thrombus removal direction and the tip to be parallel to each other during the thrombus aspiration.

In addition, the aspiration catheter of the present disclosure may shorten the thrombus resection procedure time and increase the success rate due to the improved thrombus aspiration efficiency during the thrombus resection because the catheter may be steered and expanded simultaneously by pulling the wire, which is the drive part, or by using the magnetic torque or strength of the magnetic body.

The spirit of the present disclosure should not be limited to the embodiments described above. The present disclosure may be applied to various fields and may be variously modified by those skilled in the art without departing from the scope of the present disclosure claimed in the claims. Therefore, it is obvious to those skilled in the art that these alterations and modifications fall within the scope of the present disclosure.

What is claimed is:

1. A steerable and expandable aspiration catheter comprising:
a body;
a steering part positioned at an end of the body and steering the end;
an expanding part connected to an end of the steering part and expanding a diameter of the end; and
a power unit providing power for the steering and the expansion,
wherein the steering part includes a first body having a circular ring shape and a leg connecting the first body and the expanding part to each other,
the expanding part includes a second body having a circular ring shape and a plurality of tip parts each having an annular shape, each protruding outward from the second body, and arranged in a radial direction, and
the power unit includes a plurality of wires, each wire being connected to each tip part, and controls the steering of the tip part or expands a diameter of the tip part based on a displacement of the wire pulling the tip part in a direction opposite to an end of the catheter.

2. The catheter of claim 1, wherein the steering part includes a pair of a 1-1-th body and a 1-2-th body each having a circular ring shape and spaced apart from each other, a first leg connecting the 1-1-th body and the 1-2-th body to each other, and a second leg connecting the 1-2-th body and the second body to each other,
one pair of the first legs or one pair of the second legs are disposed to face each other, and
a first line connecting the pair of first legs to each other and a second line connecting the pair of second legs to each other are orthogonal to each other.

3. The catheter of claim 1, wherein the power unit controls the steering of the tip part of the catheter by generating the displacement between one pair of wires disposed on one side of the wires and one pair of wires disposed on the other side of the wires.

4. The catheter of claim 1, wherein the power unit expands the diameter of the tip part as each tip part is bent outward in the radial direction when all of the wires are pulled to generate no displacement difference between the respective wires.

5. The catheter of claim 4, wherein in the catheter, the diameter of the tip part is controlled by a displacement as much as the amount that the wire is pulled when all of the wires are pulled.

6. The catheter of claim 4, wherein the tip part includes
a tip part body having an annular shape and protruding outward from the second body while being spaced apart therefrom by a certain distance, and
a tip part supporter made of a flexible material, having a linear shape, connecting the tip part body and the second body to each other, and supporting the tip part body.

7. A steerable and expandable aspiration catheter comprising:
a body;
a steering part made of a magnet and positioned at an end of the body;
an expanding part made of a combination of a plurality of magnets and positioned at an end of the steering part; and
a magnetic field generating unit generating a magnetic field around the steering part and the expanding part,
wherein the steering part controls steering of the steering part based on a direction of the magnetic field, and the expanding part controls a diameter of an end of the catheter based on a strength of the magnetic field.

8. The catheter of claim 7, wherein the steering part is a circular base magnet and has polarities disposed in a length direction of the catheter, and
the base magnet steers the end of the catheter based on the direction of the magnetic field.

9. The catheter of claim 8, wherein the expanding part includes a tip magnet part disposed outside an end of the base magnet to be spaced apart therefrom and a leg connecting the tip magnet part and the steering part to each other,
the tip magnet part is a combination of a plurality of arc-shaped magnets, has polarities disposed in a radial direction, and is displaced outward in a radial direction based on a strength of the magnetic field generated around the expanding part to expand a diameter of the end of the catheter.

10. The catheter of claim 1, wherein the catheter further includes a catheter for pressure measurement positioned on the outside of the body and capable of measuring a surrounding pressure.
